(19) United States
(12) Patent Application Publication
(10) Pub. No.: US 2019/0290623 A1
(43) Pub. Date: Sep. 26, 2019

(54) USE OF TIOTROPIUM BROMIDE TO PREVENT PULMONARY OXYGEN TOXICITY

(71) Applicant: The Unites States of America Represented by the Secretary of Navy, Silver Spring, MD (US)

(72) Inventors: Aaron A. Hall, Clarksburg, MD (US); William A. Cronin, Chevy Chase, MD (US); Richard T. Mahon, Germantwon, MD (US); William R. Johnson, Washington, DC (US)

(21) Appl. No.: 16/365,364

(22) Filed: Mar. 26, 2019

Related U.S. Application Data
(60) Provisional application No. 62/648,248, filed on Mar. 26, 2018.

(51) Int. Cl. A61K 31/439; A61K 31/40; A61K 31/46; A61P 11/08

(52) U.S. Cl. CPC A61K 31/439; A61P 11/08; A61K 31/46; A61K 31/40

(57) ABSTRACT

A method for the prevention of the onset of pulmonary oxygen toxicity and/or reduction of decrements of pulmonary function due to pulmonary oxygen toxicity comprising administration of a prophylactically effective amount of an anticholinergic, optionally together with a pharmaceutically acceptable excipient.

### CROSS-REFERENCE TO RELATED APPLICATION

This application claims benefit to U.S. provisional application No. 62/648,248 filed on Mar. 26, 2018.

### FIELD OF THE INVENTION

This invention relates to a novel use of tiotropium bromide in prevention of pulmonary oxygen toxicity.

### BACKGROUND

Pulmonary toxic effect of oxygen can arise after prolonged exposure to oxygen greater than 0.5 ATA (atmosphere absolute). Clinical features of pulmonary oxygen toxicity can be divided into three phases (a) Tracheobronchitis (b) exudative phase (c) proliferative phase. Tracheobronchitis is characterized by a reduction in pulmonary vital capacity, and the onset of symptoms including: fatigue, burning upon inspiration, and cough. Exudative phase is characterized by type 1 pneumocyte destruction, type 2 pneumocyte hyperplasia, pulmonary edema, and hyaline membrane formation. Proliferative phase is characterized by immune cell infiltration, pulmonary fibrosis, and hemorrhage. Exposure to high partial pressures of oxygen at surface level (i.e., 1 ATA) causes progressive lung injury over days that appears to be rooted in the generation of reactive oxygen species (ROS), inflammation, and eventually alveolar capillary barrier breakdown with little if any influence on the central nervous system (CNS). The first signs of toxicity appear in human after about 10 hours of oxygen at 1 ATA.

In contrast, lung injury caused by hyperbaric (HBO) oxygen exposure develops in hours, and may have CNS mediated injury based on the “depth” of exposure involving the sensory nerves that innervate the airways [5]. ROS are generated rapidly during HBO exposure, and can stimulate the aforementioned sensory nerves by interacting with redox sensitive domains of transient receptor cation channels (TRP) receptors [6]. This stimulation likely causes the early tracheobronchitis that is observed in pulmonary oxygen toxicity (PO2T). Continued stimulation of these sensory nerves activates vagal derived parasympathetic afferent nerve fibers, which cause bronchoconstriction, and increased mucus secretion leading to reversible decreases in pulmonary function. Studies at the Navy Experimental Diving Unit demonstrated that more than 40% of divers had objective findings of diminished pulmonary function after a six hour in-water dive at 1.3 ATA. Also, 33% of subjects in that study complained of symptoms such as cough, tracheal burning and chest tightness [2]. Not always in association with the overt symptoms, almost 60% of the divers showed adverse effects of the dives.

Due to the risks of pulmonary oxygen toxicity, military divers breathing enriched oxygen mixtures are limited in their diving durations. For instance, the US Navy Dive Manual limits the use of the MK16 MOD 1 closed circuit oxygen rebreather to 240 minutes per day, and 16 hours per week to specifically minimize incident of PO2T. The lack of a FDA approved pharmacologic agent, which can mitigate PO2T, remains a critical capability gap. PO2T has the potential to translate into decreased physical performance during crucial operational tasks, and limits overall mission durations.

This invention describes a method for preventing the onset, and development of pulmonary oxygen toxicity due to oxygen exposure. The inventive method is specially directed to the prevention of tracheobronchitis caused by oxygen exposure.

### DETAILED DESCRIPTION OF DRAWINGS

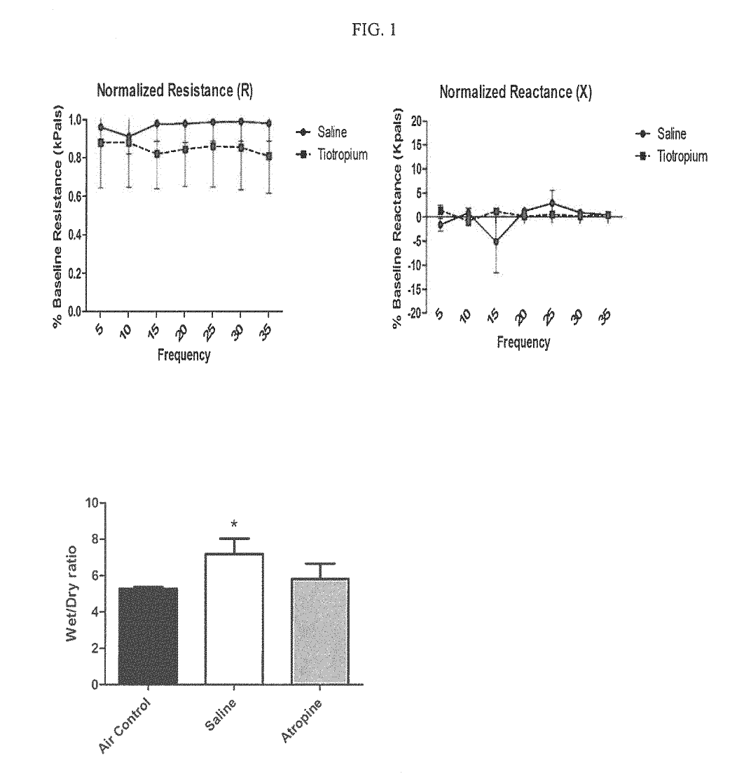

FIG. 1 shows Impulse Oscillometry (IOS) testing results for shallow water diving stimulation. A total of 14 swine (n=7/treatment arm) had successful measurements, and the post-dive data was normalized to the baseline measurement. There were no significant differences observed at any frequency in resistance (p=0.172) or reactance (p=0.65) between Tiotropium treated animals and saline controls (Two-way ANOVA with post-hoc Bonferroni's test).

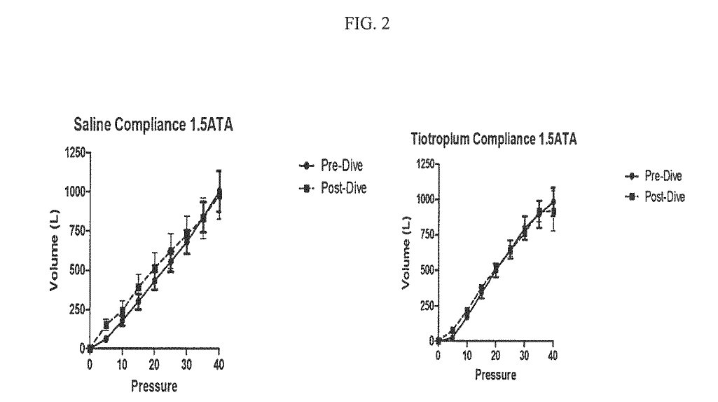

FIG. 2 shows baseline and post-dive assessments of pulmonary compliance for shallow water diving stimulation. A total of 12 swine (n=5 Saline, n=7 Tiotropium) had successful measurements. No significant changes in compliance between baseline and post-dive assessments were observed in Saline (p=0.3626) or Tiotropium (p=0.8364) treated animals (Two-way ANOVA with post-hoc Bonferroni's test).

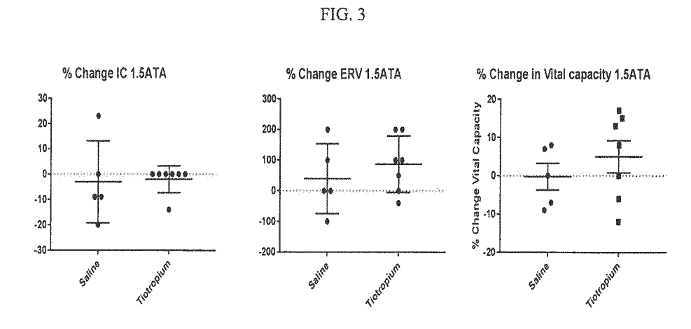

FIG. 3 shows baseline and post-dive assessments of pulmonary capacity shallow water diving stimulation. A total of 12 swine (n=5 Saline, n=7 Tiotropium) had successful measurements. No significant differences between Saline and Tiotropium treated animals were observed in: Inspiratory capacity (p=0.879), Expiratory Reserve Volume (p=0.445), or Vital Capacity (p=0.394) (Student's t-test). These values were expressed as percent change from baseline.

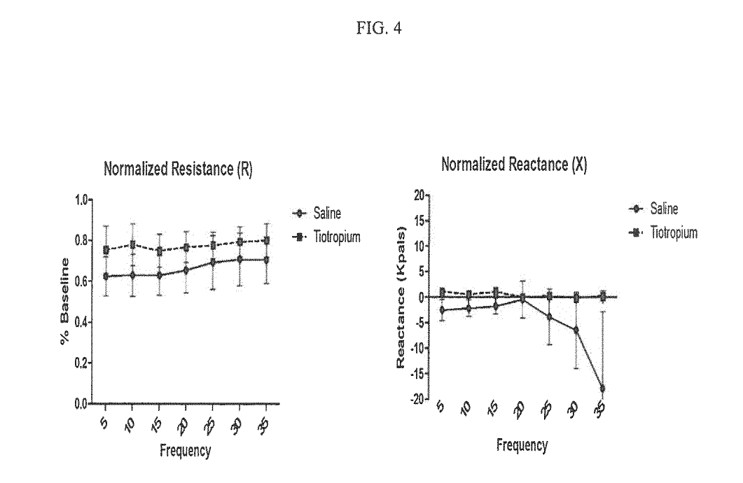

FIG. 4 shows the baseline and post-dive IOS measurements for deep diving stimulation. A total of 12 swine (n=6/treatment arm) had successful measurements, and the post-dive data was normalized to the baseline measurement. There was a significant decrease in resistance observed at all frequencies (p=0.0421) and a decrease in reactance which approached significance (p=0.0587) in saline treated controls when compared to Tiotropium treated swine (Two-way ANOVA with post-hoc Bonferroni's test).

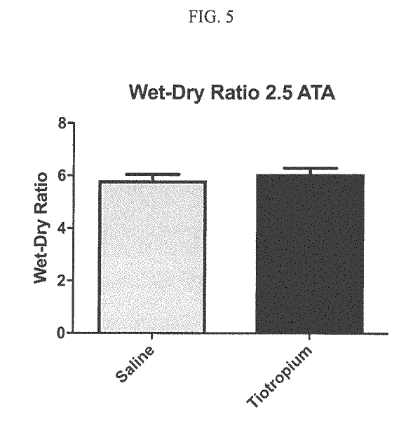

FIG. 5 shows wet/dry ratio of swine exposed to 2.5 ATA oxygen for 5.5 hrs.

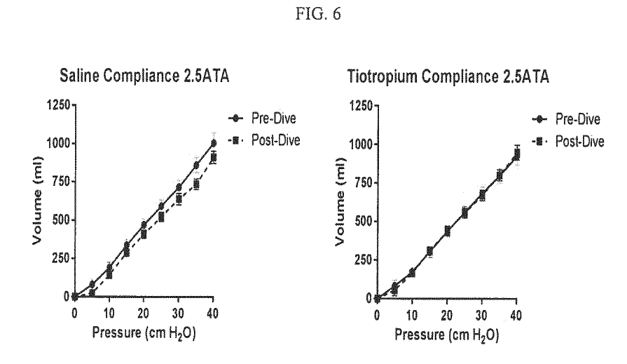

FIG. 6 shows exposure to hyperbaric oxygen (pO2>0.90) at 2.5 ATA for 5.5 hrs induced a significant decrement (p=0.0003) in pulmonary compliance in saline treated controls. Swine treated prophylactically with 18 μg Tiotropium (inhaled, nebulized) exhibited no decrement in pulmonary compliance (p=0.9094).

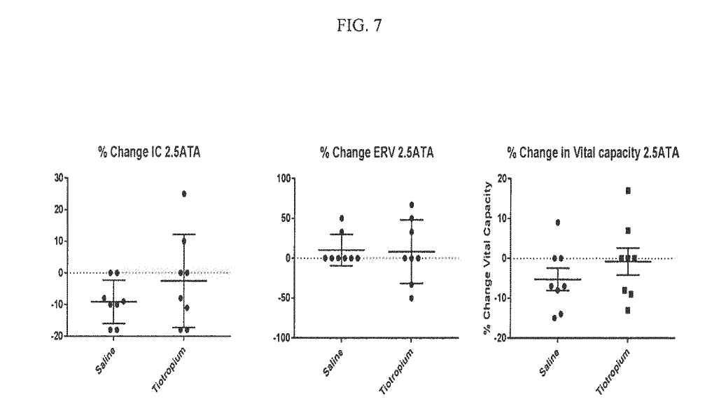

FIG. 7 shows that averaged pulmonary capacities were not significantly changed due to 2.5 ATA hyperbaric oxygen exposure. A total of 17 swine (n=8 Saline, n=9 Tiotropium) had successful measurements and values were expressed as percent change in baseline. No significant differences between Saline and Tiotropium groups were observed in: Inspiratory capacity (p=0.266), Expiratory Reserve Volume (p=0.900), or Vital Capacity (p=0.325) (Student's t-test).

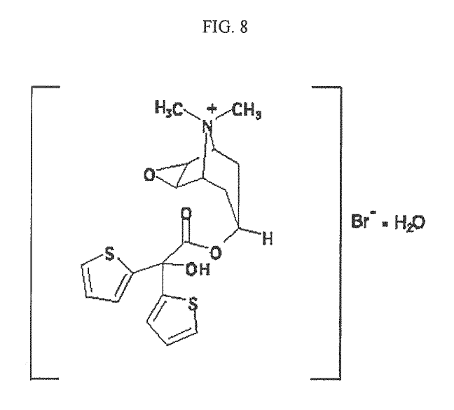

FIG. 8 shows structure formula for tiotropium bromide.

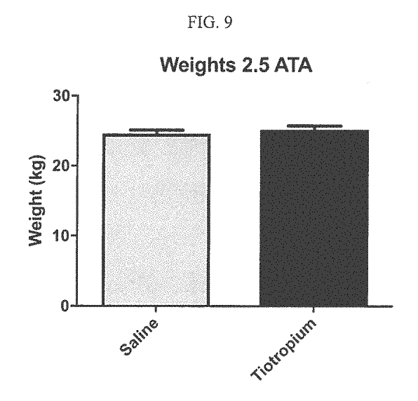

FIG. 9 shows weights of swine between the saline and tiotropium groups in deep diving experiment.

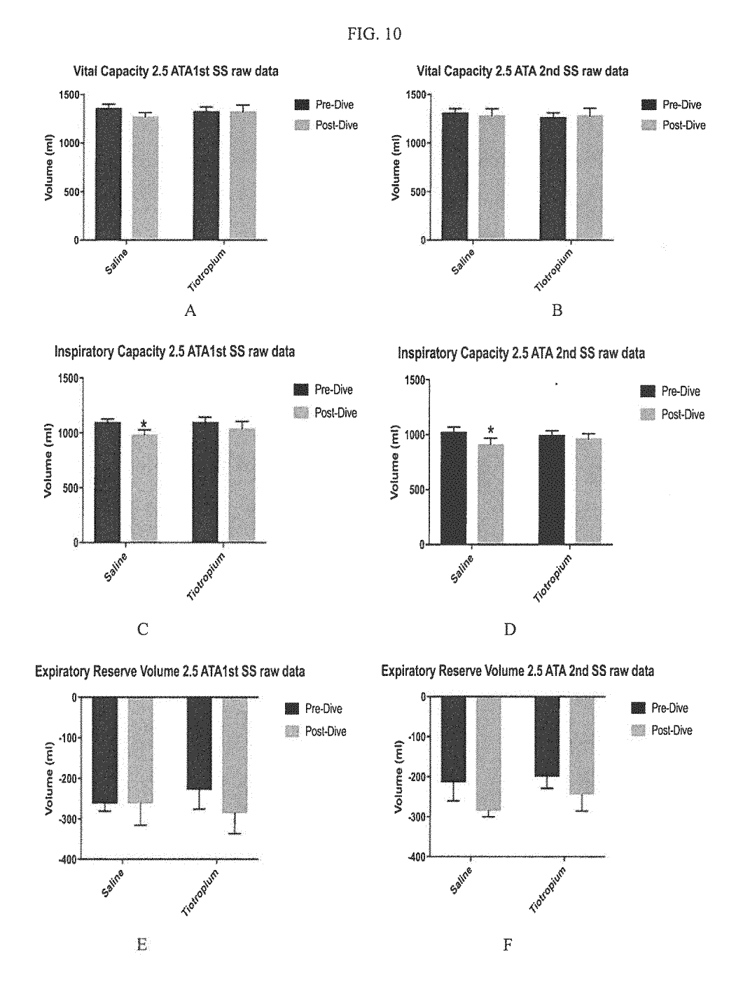

FIG. 10 shows baseline and post-dive assessments of lung capacity of swine exposed to 2.5 ATA oxygen for 5.5 hrs correcting for multiple measurements.

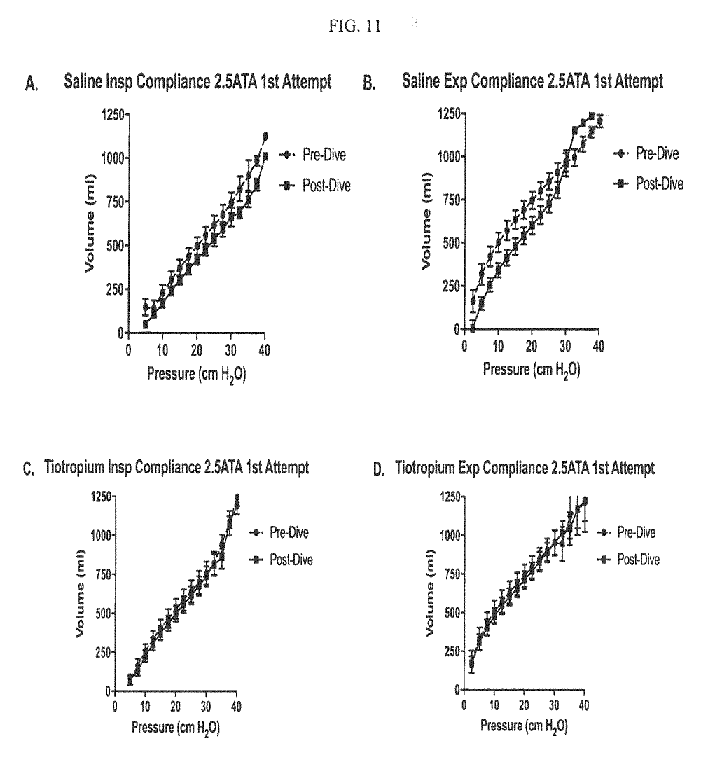

FIG. 11 shows baseline and post-dive assessments of lung compliance of swine exposed to 2.5 ATA oxygen for 5.5 hrs correcting for recruitment effects (1st measurement).

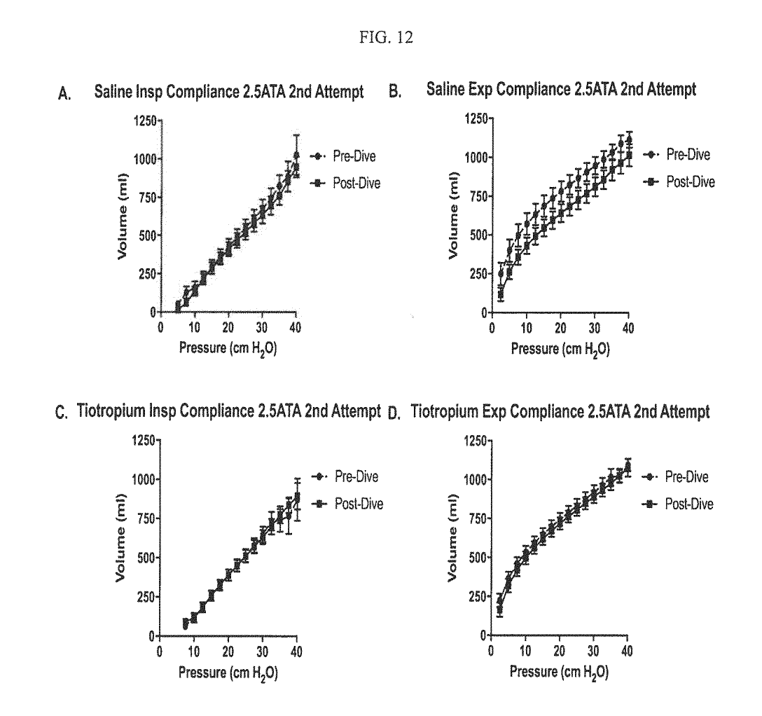

FIG. 12 shows baseline and post-dive assessments of lung compliance of swine exposed to 2.5 ATA oxygen for 5.5 hrs (2^{nd} measurement).

### SUMMARY OF INVENTION

The present invention is directed to a method for preventing pulmonary oxygen toxicity due to oxygen exposure, which comprises administering to a subject a prophylactically effective amount of an anticholinergic or pharmaceutically acceptable salts thereof, optionally together with a pharmaceutically acceptable excipient prior to an oxygen exposure. The anticholinergic or pharmaceutically acceptable salts include but not limited to tiotropium salts, oxitropium salts, flutropium salts, ipratropium salts, glycopyrronium salts and trospium salts, such as tiotropium bromide, oxitropium bromide, glycopyrronium bromide or ipratropium bromide. The anticholinergic or pharmaceutically acceptable salts thereof is administered in one or more dose prior to each oxygen exposure via oral inhalation, and is preferably administered within 24 hours prior to an oxygen exposure. The present invention prevents the onset of tracheobronchitis and decrements in pulmonary compliance due to PO2T.

### DETAILED DESCRIPTION OF INVENTION

A muscarinic receptor antagonist, is often referred to as an antimuscarinic or anticholinergic agent. Anticholinergic agents, such as tiotropium bromide, can directly block parasympathetic afferent nerve fibers through selective muscarinic antagonism. Although tiotropium bromide (FIG. 8) does not display selectivity for specific muscarinic receptors, when topically applied, it acts mainly on M3 muscarinic receptors [8] located on smooth muscle cells and submucosal glands. This leads to a reduction in smooth muscle contraction, and mucus secretion, and thus produces a bronchodilatory effect. Interestingly, tiotropium bromide is also known to reduce airway reactivity, and cough by inhibiting transient receptor potential (TRP) channels. As such, tiotropium bromide makes a unique and promising candidate for the prevention of PO2T owning to its ability to target both the TRP, and anti-muscarinic receptors. Originally marketed as SPIRIVA®, tiotropium bromide is a long-acting, 24-hour, anticholinergic bronchodilator used in the management of chronic obstructive pulmonary disease (COPD) and asthma. Clinically, tiotropium bromide has gained widespread use for the treatment, and management of chronic obstructive pulmonary disease (COPD), asthma and chronic cough in COPD patients. In addition to its clinical efficacy, tiotropium bromide has shown to have a favorable side effect profile. Currently tiotropium bromide is an FDA approved drug that is readily available in civilian and military clinics.

An aspect of this invention is a method for preventing the onset and development of pulmonary oxygen toxicity due to oxygen exposures comprises of administrating to a subject one or more doses of a prophylactically effective amount of an anticholinergic, optionally together with a pharmaceutically acceptable excipient. The inventive method is specially directed to the prevention of tracheobronchitis or decrement of pulmonary function due to oxygen exposure, such as in decompression therapy, hyperbaric oxygen therapy, and diving. The anticholinergic may be selected from a group consisting of tiotropium salts, oxitropium salts, flutropium salts, ipratropium salts, glycopyrronium salts and trospium salts. Examples of such salts include but not limited to tiotropium bromide, oxitropium bromide, glycopyrronium bromide and ipratropium bromide. In a preferred embodiment, tiotropium bromide is given to a subject within 24 hours of an anticipated oxygen exposure via nasal or oral respiratory route administration. The administration may start days before the anticipated oxygen exposure. For example, a diver may take an oral inhalation of tiotropium bromide within 24 hours of an anticipated hyperbaric oxygen exposure (i.e. hour 24-0 before a dive or decompression therapy session) to prevent the onset and development of PO2T. Alternatively, prophylactic effective dose of tiotropium bromide may be taken daily for several days prior to an oxygen exposure. The administration may be repeated before each oxygen exposure. The claimed method may be also used to reduce decrements in pulmonary function due to PO2T, such pulmonary functions include but not limited resistance, reactance, pulmonary compliance and pulmonary capacity.

Animal studies performed at NMRC showed efficacy of anticholinergic or its pharmaceutically acceptable salts as a prophylactic to prevent PO2T. The following swine studies were designed to emulate operational exposures (and associated pulmonary function decrements) encountered during the course of diving operations and to assess the efficacy of Tiotropium Bromide prophylaxis at reducing decrements in pulmonary function secondary to PO2T and preventing tracheobronchitis associated with oxygen exposure.

### Example 1: Pulmonary Effect of Tiotropium and Hyperbaric Oxygen in Swine

Experiment designs are summarized in Table 1.

TABLE 1
Experimental Design for swine operational exposure simulation

| Variables | Experiment 1 | | Experiment 2 | |
|---|---|---|---|---|
| Animal Group | Control | Experimental | Control | Experimental |
| | (n = 10) | (n = 10) | (n = 14) | (n = 14) |
| Anesthesia | Yes | Yes | Yes | Yes |
| Dive Profile | 1.5ATA for 17.5 hr | | 2.5ATA for 5.5 hr | |
| Therapy | Normal Saline | Tiotropium | Normal | Tiotropium |
| | | bromide | Saline | bromide Pre |
| | | Pre-Dive | | Dive (18 mcg) |
| | | (18 mcg) | | |

| | |
|---|---|
| Data Collection | Pulmonary function tests and post- |
| | mortem histological samples |

### Experiment 1: Shallow Operational Exposure Simulation

Materials and Method

Experiment 1 is designed to evaluate the efficacy of tiotropium bromide administered pre-dive in reducing decrements in pulmonary function due to PO2T following a 17.5 hour, 1.5 ATA hyperbaric oxygen exposure. This dive profile was selected because it was of sufficient duration to elicit persistent pulmonary function decrement in humans [3].

Swine (20 kg, n=20) were removed from their run, placed in a Panepinto sling, pre-medicated with Diazepam (15 mg IM) and had a baseline pulmonary function assessment via impulse Oscillometry. The swine were anesthetized with propofol (20 cc, I.V. target, adjusted as needed to optimize sedation), intubated and ventilated. Anesthetized swine were shaved, then ECG and EEG electrodes were placed. Animals were then paralyzed with Atracurium Besylate (50 mg, IV) for accurate pulmonary function measurements of quasi-static and dynamic compliance.

Following baseline pulmonary function measurements, the swine were randomized to receive equivalent volumes of normal saline or tiotropium bromide (18 mcg) via nebulizer. The swine were then ventilated until muscle tone was regained. Sedation was discontinued and the swine were extubated, transported to the hyperbaric chamber and allowed to recover until able to independently stand. Once able to stand, animals had electrode leads attached and IV tubing connected to the ear vein catheter. The chamber hatch was then closed and the swine compressed to 1.5 ATA on air.

Next, the swine were switched to 100% oxygen breathing gas and remained at 1.5 ATA for 17.5 hr. At the end of the 17.5 hour 1.5 ATA oxygen exposure, swine surfaced at a rate of 1 ATA/minutes (ATA/min), were transferred to a Panepinto sling, sedated with diazepam (1.5-2.0 mg/kg, I.V) and reassessed via impulse Oscillometry. The swine were again anesthetized with propofol and intubated. When fully sedated, the swine were ventilated, paralyzed with Atracurium Besylate, and post-dive measurements of pulmonary function were recorded. During all phases of study the swine were continuously monitored for signs of seizure or distress.

Once measurements were complete, animals were then euthanized by intravenous infusion of euthanasia solution (Euthasol 1-1.5 ml/10 lbs. body weight) and transported to the necropsy suite for post-mortem sample collection. CNS oxygen toxicity did not occur with this dive profile.

### Results

A) Pulmonary Function Testing: Impulse Oscillometry (IOS)

IOS testing is a noninvasive pulmonary function test designed to provide data on pulmonary function by introducing oscillating pressure waves at varying frequencies into the airways (see Komarow et al. [4] for review). The two measurements of interest for this study are resistance (R) and reactance (X). Resistance is defined as the energy required to propagate an air pressure wave through the airways and is a measure of airway caliber. Reactance is a measurement of inertia and elasticity of the air ways and lung tissue. Baseline and post-dive IOS measurements were conducted on each animal enrolled in the study. A total of 14 swine (n=7/treatment arm) had successful measurements, and the post-dive data was normalized to the baseline measurement. As shown in FIG. 1, there were no significant differences observed at any frequency in resistance (p=0.172) or reactance (p=0.65) between Tiotropium treated animals and saline controls (Two-way ANOVA with post-hoc Bonferroni's test).

B) Pulmonary Function Testing. Pulmonary Compliance

Baseline and post-dive assessments of pulmonary compliance were conducted on each animal enrolled in the study. A total of 12 swine (n=5 Saline, n=7 Tiotropium) had successful measurements. As shown in FIG. 2 there is no significant changes in compliance between baseline and post-dive assessments were observed in Saline (p=0.3626) or Tiotropium (p=0.8364) treated animals (Two-way ANOVA with post-hoc Bonferroni's test).

C) Pulmonary Capacities

Baseline and post-dive assessments of pulmonary compliance were conducted on each animal enrolled in the study. A total of 12 swine (n=5 Saline, n=7 Tiotropium) had successful measurements. As shown in FIG. 7, there is no significant differences between Saline and Tiotropium groups were observed in Inspiratory capacity (p=0.879), Expiratory Reserve Volume (p=0.445), or Vital Capacity (p=0.394) (Student's t-test). These values were expressed as percent change from baseline.

Anticholinergic therapy reduces pulmonary edema in swine subjected to 72 hours of normobaric hyperoxia. *Denotes p<0.05 as determined by one-way ANOVA with post-hoc Dunnet's test.

TABLE 2
Weight and Wet/Dry ratio

| | Saline (n = 9) | Tiotropium (n = 9) | p Value |
|---|---|---|---|
| Weight (SD) | 24.58 (5.388) | 27.51 (4.538) | P = 0.6195 |
| Wet/Dry Ratio | 5.858 (0.779) | 5.876 (0.491) | P = 0.9526 |
| (SD) | | | |

### Discussion

There were no observed decrements in pulmonary function in swine exposed to 1.5 ATA for 17.5 hours using IOS or direct compliance measurement. This is in contrast to the observed decrements in humans reported by Clark et al. [3]. A limitation of using swine for this study is the lack of cooperation with the subject, which limits the use of spirometry, and a lack of communication preventing the reporting of discomfort. Forced vital capacity, the main spirometry endpoint reported to be altered with pulmonary oxygen toxicity, could in fact be impacted by the discomfort known to occur with pulmonary oxygen toxicity. Human studies utilizing IOS will likely be useful in further parsing the relative contributions of patient discomfort, and pulmonary function decrements during early stage pulmonary oxygen toxicity. Overall, within this arm of the study tiotropium had no observable adverse effect on outcome at any of the assessed endpoints.

Experiment 2: Deep Water Operational Exposure Simulation

Materials and Method

Experiment 2 is designed to evaluate the efficacy of tiotropium bromide pre-dive with respect to reducing decrements in pulmonary function due to PO2T following a 5.5 hour, 2.5 ATA hyperbaric oxygen exposure. This dive profile was selected because it caused the most significant pulmonary function deficits in humans [3] with minimal risk of seizure due to CNS oxygen toxicity.

Twenty-eight male castrated Yorkshire swine (24.88+1.959 kg) were housed in at the animal care facility on site for 5 days before any procedures. They were fed 2-2.5% of body weight twice daily (LAB DIET® Mini-Pig Grower, Quality Lab Products, Elkridge, Mass.) and water was provided ad lib.

Nebulizer Solution Preparation: Tiotropium bromide (SPIRIVA®) dry powder capsules were opened and the contents dissolved in normal saline solution (0.9% NaCl, Hospira Inc., Lake Forest Ill.) and filter sterilized using a 0.221 m filter prior to administration similar to that previously described.

On the day of HBO exposure, the animals were placed in a Panepinto sling and had an ear vein cannulated using an 18-22 gauge angiocath catheter system. The swine was then induced with intravenous propofol (20 ml, 10 mg/ml) (Emergency Medical Products INC, Cudahy, Wis.) and intubated. Sedation was maintained via a continuous propofol drip (1.0 mg/min, IV), and the animal was ventilated on volume control mode with the following settings: Volume=10 cc/kg, I:E=1.4, RR=16, PEEP=5.0 with a fraction of inspired O2 (FiO2) of 1.0.

The swine were prepared for ECG recording as described previously. Briefly, using an electric shaver, ECG areas were prepared bilaterally, posterior to the shoulder, lateral to the vertebrae, at approximately 4-7 cm above ribs 5-7 (bipolar derivation). Another area was prepared 2 cm lateral to the vertebrae on right side, between ribs 12-13. Newly exposed skin was rubbed with isopropyl alcohol. Disposable pre-wired (Grass Technologies/Astro-Med, Inc. Warwick, Mass.) or SF405 (KENDALL MEDI-TRACE™, Tyco Healthcare, Mansfield, Mass.) self-adhesive Ag/AgCl electrodes were placed on the proximal, symmetric sites and the ground electrode was placed at the distal site (between ribs 12-13). The animal was then held for 5 minutes to capture baseline ECG recordings.

Pulmonary function testing: The swine had disposable prewired electrodes adhered to the dermis over the gluteal muscle and a train of four (TOF) stimulation was initiated to confirm proper placement and twitch using a DigiStim III Peripheral Nerve Stimulator (Neuro Technology Inc., Kerrville, Tex.). Atracurium besylate (50 mg, IV) (hospira, Lake Forest, Ill.) was injected into the ear vein catheter, and the swine was observed until cessation of muscle twitch with TOFs. The swine was then disconnected from the ventilator circuit, allowed to passively exhale, and connected to an O₂ filled 3 L super syringe (Hamilton Company, Reno, Nev.) attached to a pressure gauge (OMEGA®, Norwalk, Conn.), and pneumotachometer (Harvard Apparatus, Holliston, Mass.). Oxygen was introduced in 100 ml increments every 3 seconds until a static pressure of 40 cm H₂O was reached. The lungs were then deflated in 100 ml increments until a static pressure of −40 cm H₂O. The swine was then reconnected to the ventilator and ventilated for 2 minutes before repeating the insufflation and exhalation maneuvers and measurements one more time. After these lung pressure-volume measurements, the swine were maintained on ventilator support until a return of muscle twitch was observed with the TOF stimulation, and the swine could breathe spontaneously with the ventilator on manual mode. After the above measurements, swine received either nebulized Tiotropium (18 μg dissolved in saline) or an equal volume of 0.9% saline through the breathing circuit over the course of 15 minutes. The propofol infusion was then stopped, the animals were then monitored until a swallow reflex was observed then extubated. The swine were then transported to the hyperbaric chamber suite and allowed to recover until capable of standing.

Hyperbaric Exposures: Individual animals were then placed into a Plexiglas box (30 in.×42 in.×38 in., manufactured in-house) within a Multiple Large Animal Chamber (MLAC), which is a steel-hulled hyperbaric chamber (450-cubic ft. floodable volume and pressure tested to 1,230 feet of seawater [fsw] equivalent) (Bethlehem Steel Corp, Bethlehem Pa.). Pressure/depth was monitored by a CPG 2500 Digital Pressure Gauge (Mensor Corp., San Marcos, Tex.). Swine were dove in pairs with each treatment group represented. Swine box assignment was alternated with respect to treatment to avoid any effects associated with individual box environmental features. Animals were monitored via closed-circuit television to observe signs of distress related to middle ear barotrauma (e.g. head shaking, nystagmus) and seizure activity. Oxygen concentration was measured and recorded using a portable O2 analyzer (Teledyne Analytical Inst, City of Industry, Calif.). After positioning the animal inside the Plexiglas box, the MLAC was sealed and pressurized using air at the rate of I ATA/minute to a depth of 2.5 ATA. After 2.5 min at a bottom depth equivalent to 2.5 ATA, the breathing gas was switched to 100% O₂ and maintained for 5.5 Hrs. After completing 5.5 Hrs on 100% O2, the inspired gas was switched back to air for 3 min. Then, the chamber was decompressed at 1 ATA/minute. Upon reaching surface the animal was transported to the surgical suite for post-dive ECG and pulmonary function measurements (identical to the pre-dive procedures). The animal was then euthanized via ear vein infusion of Euthasol (1.0-1.5 ml/10 lbs body weight) followed by a saline flush (5 cc).

Lungs were collected from the swine during necropsy. The right lung was collected for measurement of wet-dry ratios. The left lung was fixed via tracheal instillation at a standard pressure of 25 cmH₂O with 10% neutral buffered formalin (NBF). The lungs were then sectioned into ten, one centimeter (cm) cranial to caudal serial sections. Three, 1 cm blocks were cut from each section and post-fixed in 10% NBF, trimmed, embedded in paraffin and cut into 5 μm sections for histological staining. Histology slides were de-paraffinized and stained with hematoxylin and eosin. A board-certified veterinary pathologist blinded to group randomization reviewed all gross necropsies, images, and histology slides.

Wideband AC amplifiers (Grass, Quincy, Mass.) were used for EEG and ECG recordings; a 0.5-35 Hz and 60 cycle notch filter setting was used. The EEG signal was collected by a Dell Laptop (Round Rock, Tex.) and an analog-digital converter card (PCI-6052E, National Instruments, Austin, Tex.) with 16 bit resolution and 200 Hz sampling rate. Heart rate variability (HRV) was calculated from pre-dive ECG epochs of at least 2 min durations using the Labchart ECG tool suite. In this case, ECG R-waves were used for trigger peak detection in the software. Above the manually set trigger level the LabChart software looked for maximum values and measured the time interval between peaks (maximal amplitude of R-wave). The time interval between peaks was referred to as the NN interval. The result is given as a frequency distribution histogram, peak-to-peak graph as well as numbers for the following values: 1) number of peaks found; 2) mean; 3) standard deviation (SD); 4) median; 5) minimum; and 6) maximum. To characterize HRV, we used SD values of each group. The length of measured periods were: (1) 114.995 sec; peak-to-peak distance 0.750 t 0.0004 sec; 153 peaks; FFT 1.329 Hz=79.74 bpm; and (2) 326.52 sec; peak-to-peak distance 0.750±0.0003; 434 peaks; FFT 1.332 Hz=79.92 bpm.

Pulmonary function data analysis: Pressure and flow data from the pressure gauge and pneumotachometer respectively were collected by a Dell Laptop (Round Rock, Tex.) and an analog-digital converter card (PCI-6052E, National Instruments, Austin, Tex.) with 16 bit resolution and 200 Hz sampling rate running Labchart 8.0. Volume data was extrapolated using the Spirometry analysis module with Labchart. Pressure values from the 30 sec interval prior to the next introduction of oxygen, via 3 L super syringe, were averaged and plotted against the insufflation volumes. These data were transferred into an excel spreadsheet for analysis. Inspiratory capacity (IC), Expiratory Reserve Volume (ERV), and Vital capacity (VC) measurements were collected from the raw pressure volume loop data. Pressure volume loop data sets collected from the first and second syringe procedures were analyzed separately to take into account that the 1st procedure may act as a recruitment maneuver, opening collapsed alveoli. IC was defined as the total volume from 0 to 40 cm H₂O pressure. ERV was defined as the volume from 0 to −40 cm H₂O. VC was defined as the sum of the IC and the absolute value of the ERV. Lung compliance measurements were taken from each arm of the extrapolated pressure volume data. Similar to the lung volume analysis, pressure volume loop data sets collected from the first and second syringe procedures were analyzed separately.

All data were compiled into Excel spreadsheets then imported into Graphpad Prism 7.02 for statistical analysis. Each animal had a different lung volume required to achieve a pressure of 40 cm H₂O. To allow for accurate data comparison between animals and/or groups the pressure volume curves were separated into inspiratory arms (0 to 40 cm H₂O) and expiratory arms (40 to −40 cm H₂O). Each arm was then fit using a non-linear regression utilizing a Gaussian distribution model. Volume data was then extrapolated from the curve corresponding to pressures from 0 to 40 cmH₂O at 2.5 cm H₂O increments. Statistical group-wise comparison of the extrapolated inspiratory and expiratory arms were conducted using repeated measures two-way ANOVA. Lung volume data was compared using two-way ANOVA with post-hoc Sidak's multiple comparison test. Statistical comparison of weight and wet/dry data was conducted using a 2-tailed Student's t-test, and HRV comparison was conducted using One-tailed Student's T-test. All data is presented as mean t SEM Results

As shown in FIG. 9, weights were not different between swine in the saline (24.59±0.504 kg, n=14) and tiotropium (25.16±0.550 kg, n=14) groups (p=0.4462, two-tailed students t-test). Similarly, Wet-Dry ratios of lung collected from saline (5.825±0.2227, n=13) and tiotropium groups (6.067±0.2305, n=13) were not significantly different (p=0.4566, two-tailed students t-test) (FIG. 5). There was one incidence of seizure in the saline group resulting in a dive abortion, which reduced the number in each group by one.

TABLE 3
Weight and Wet/Dry ratio

| | Saline (n = 14) | Tiotropium (n = 14) | p Value |
|---|---|---|---|
| Weight (SD) | 24.59 (1.886) | 25.16 (2.058) | P = 0.4462 |
| Wet/Dry Ratio (SD) | 5.825 (0.222) | 6.067 (0.230) | P = 0.4566 |

Lungs in all groups did not have gross or histological evidence of edema, or other injuries associated with the exudative phase of pulmonary oxygen toxicity.

A) Impulse Oscillometry (IOS)

Baseline and post-dive IOS measurements were conducted on each animal enrolled in the study. A total of 12 swine (n=6/treatment arm) had successful measurements and the post-dive data was normalized to the baseline measurement. As shown in FIG. 4, there was a significant decrease in resistance observed at all frequencies (p=0.0421) and a decrease in reactance, which approached significance (p=0.0587) in saline treated controls when compared to Tiotropium treated swine (Two-way ANOVA with post-hoc Bonferroni's test).

B) Pulmonary Capacity:

The analysis of the 1st set of pulmonary function measures showed that VC decreased in the saline group following hyperbaric exposure (87.5+46.9 ml, p=0.1624, n=8) whereas the tiotropium group had no change (FIG. 10 panel A). During the 2nd set of pulmonary function measurements, the saline group had decreased VC (28.57±59.75 ml, p=0.8703, n=7) while the tiotropium group showed an increase in VC (22.22+52.69 ml, p>0.8974, n=9) (FIG. 10 panel B). The analysis of the 1st set of measurements showed that IC decreased in the saline group following hyperbaric exposure (112.5±31.91 ml, p=0.0074, n=8) whereas the tiotropium group IC demonstrated a smaller decrease (57.14+34.11 ml, p=0.2217, n=7) (FIG. 10 panel C). During the 2nd set of measurements, the saline group showed a decreased IC (114.3±36.22 ml, p=0.014, n=7) while the tiotropium group showed a smaller decrease in IC (33.33±31.94 ml, p=0.5300, n=9) (FIG. 10 panel D). The analysis of the 1st set of measurements showed no change in ERV in the saline group following hyperbaric exposure (0±58.6 ml, p=>0.9999, n=8) whereas the tiotropium group ERV demonstrated an increase (57.14+62.65 ml, p=0.6315, n=7) (FIG. 10 panel E). During the 2nd set of measurements, the saline group showed an increased ERV (71.43±44.78 ml, p=0.2483, n=7) while the tiotropium group showed a smaller increase in ERV (44.44±39.49 ml, p=0.4806, n=9) (FIG. 10, panel F).

Overall, as shown in FIG. 7, there is no significant differences between Saline and Tiotropium groups were observed in: Inspiratory capacity (p=0.266), Expiratory Reserve Volume (p=0.900), or Vital Capacity (p=0.325) (Student's t-test). These values were expressed as percent change from baseline. Swine exposed to 2.5 ATA oxygen for 5.5 hrs showed no signs of PO2T as identified by wet/dry ratio or decrements in arterial saturation. Anticholinergic therapy did not exacerbate either endpoint (FIG. 5).

C) Pulmonary Compliance

For the inspiratory arm, in the 1st set of measurements the saline treated animals demonstrated a significant decrease in pulmonary compliance following the hyperbaric oxygen exposure when compared to baseline (p<0.0001, n=8) (FIG. 11 panel A). In contrast, tiotropium treated animals demonstrated no significant difference (p=0.1999, n=7) (FIG. 11 panel C). In the 2nd set of measurements, there was no significant change in compliance following hyperbaric exposure in either the saline (p=0.1253, n=7) (FIG. 12 panel A) or tiotropium (p=0.7388, n=9) (FIG. 12 panel C) groups. For the expiratory arm, in the 1st set of measurements, the saline treated animals demonstrated a decreased expiratory compliance following hyperbaric exposure when compared to baseline (p=0.0028, n=8) (FIG. 11 panel B). The tiotropium group did not exhibit any decrement in expiratory compliance (p=0.5711, n=7) (FIG. 11 panel D). In the 2nd set of measurements, both saline (p<0.0001, n=7) (FIG. 12 panel B) and tiotropium (p=0.0438, n=9) groups (FIG. 12 panel D) showed a significant decrement in pulmonary compliance.

D) Heart Rate Variability.

Heart rate variability was analyzed as a surrogate marker of parasympathetic tone. ECG epochs were collected prior to compliance measurements pre-dive and post-dive and the standard deviation of the NN interval was measured. Post-dive SDNN was then normalized to the pre-dive SDNN and expressed as a mean+/−SEM. Saline treated control animals SDNN increased significantly (136.4+/−23.55) when compared to Tiotropium treated animals (103.5+/−15.87) (One-tailed Student's t-test, p=0.0331).

### DISCUSSION

In the deep water simulated dive profile, we observed decrements in pulmonary function in swine exposed to 2.5 ATA for 5.5 hours using IOS and direct compliance measurements. Decrements were observed in VC and IC (6% and 10% respectively) in saline treated animals exposed to hyperbaric oxygen. With regard to lung volumes, we observed a decrease in inspiratory capacity and expiratory compliance only in the saline group. Tiotropium likely prevented decrements in lung function characteristic of PO2T through its known anticholinergic effects. A significant decrement in expiratory compliance was observed in the saline group that was not observed in the Tiotropium group Oxygen at 2.5 ATA was associated with decreased airways resistance as measured across all frequencies. This seeming improvement in resistance may in fact be a function of decreased lung compliance; where the less compliant lung serves to externally support the airway thus lowering airway resistance. This finding is congruent with our finding of decreased compliance in the saline group and no change in Tiotropium group.

The IOS data is complex to interpret as a decrease in resistance and reactance do not fit with normal restrictive or obstructive pulmonary disease human pathology phenotypes reported (such as Asthma, COPD, Idiopathic Pulmonary Fibrosis). The decrease in resistance suggests that the large airways are increasing in caliber during the oxygen exposure, while the decrease in reactance suggests that the airways (particularly the small airways) are becoming stiffer. One plausible mechanism for this observation is an accumulation of mucus/secretions in the small airways which would decrease compliance. This would also provide an explanation as to why tiotropium prophylaxis reduced the observed changes, as reducing secretions is a well described property of this class of anticholinergics.

Importantly we observed no adverse reactions (including seizure) in the Tiotropium treated swine in either of the oxygen exposures studied. Given the potential improvement in lung function at 2.5 ATA, moving forward in human studies appears both safe and justified.

### Prophetic Example: Clinical Trial of Using Tiotropium Bromide as a Prophylactic to Prevent PO2T or to Reduce Pulmonary Function Decrements

A clinical trial is planned to evaluated tiotropium bromide's efficacy for reducing pulmonary symptom onset and pulmonary function decrements in subjects exposed to 2.0 ATA O2 for 8 hours. The O2 exposure is chosen as an operationally relevant exposure for DISSUB rescue and hyperbaric therapy. If efficacious, tiotropium bromide therapy would be applicable at the lower oxygen concentrations encountered during rebreather operations.

Study Design: The study design is a cross-over placebo controlled trial where subjects will be exposed to 2.0 ATA O2 for 8 hours on two occasions separated by a two-week washout period. The 2.0 ATA 02, 8 hour exposure profile is known to produce tracheobronchitis and an average decrement in vital capacity of 10% 6.7. There will be 30 subjects randomly assigned to one of two groups (n=15/group): tiotropium bromide or placebo prior to the first exposure. Following a two-week washout interval, the subjects that received tiotropium bromide will receive placebo and vice versa prior to the 2nd exposure. Pulmonary function testing will be conducted prior to, immediately post-dive and 24 hours post-dive for both exposures. Time to onset of symptoms will be recorded during dives as well as responses to symptom questions after the dive. Six additional subjects will be recruited (total n=36) to account for subjects lost between exposures.

| Variables | Exposure 1 | Exposure 2 |
|---|---|---|
| Subject Group | Placebo Tiotropium Bromide | Placebo Tiotropium Bromide |
| | (n = 15; Group 1) (n = 15; Group 2) | (n = 15; Group 2) (n = 15; Group 1) |
| Dive Profile | 2.0 ATA O₂ for 8 hours | 2.0 ATA O₂ for 8 hours |
| Therapy | Group 1 (Placebo) | Group 2 (Placebo) |
| | Group 2 (Tiotropium bromide, | Group 1 (Tiotropium bromide, |
| | 18 ug) | 18 ug) |
| Data Collection | Pulmonary function test and | Pulmonary function test and |
| | pulmonary symptoms | pulmonary symptoms |

Treatment Administration: Tiotropium bromide (Spiriva) or placebo will be administered using a commercial dry powder inhaler (Handihaler) at the FDA approved dose of 18 mcg (18 μg is typical human dose). Placebo Handihaler will be obtained from the manufacturer. Tiotropium bromide or placebo will be administered immediately following the baseline pulmonary function testing and prior to hyperbaric exposure.

Oxygen Exposure Profile: Subjects will be exposed to 2.0 ATA O2 for 8 hours on two occasions separated by a two-week washout period. Compression and decompression to the exposure depth will take place on air in a hyperbaric chamber located Navy's Southwest Regional Maintenance Center in San Diego, following US Navy standards. Hyperbaric medical technicians and a physician will be on site and available by remote communication and video as well as via lock-out. The two-week surface interval will allow sufficient washout of tiotropium bromide, resolution of tracheobronchitis, and non-learning of cough questionnaire.

Experimental Endpoints:

Pulmonary function testing: will follow American Thoracic Society guidelines for acquisition of expiratory flows (spirometry), lung volumes, and diffusing capacity. The primary endpoints will be forced vital capacity measurements and symptom severity with assessment as described in Clark et al 9. Secondary endpoints will include FEV1, FEV25-75, diffusing capacity (measured by DLCO), and respiratory impedance (measured by impulse Oscillometry).

Symptoms: During the exposure, subjects will have symptoms queried hourly as previously described 4. Additionally, subjects will be assessed with a modified version of the Leicester Cough Questionnaire (a standardized cough scale) after each exposure 12.

### REFERENCES

1) U.S. Navy Diving Manual, Revision 6. Naval Sea Systems Command, SS521-AG-PRO-010; April 2008.
2) Shykoff B. Pulmonary effects of eight-hour MK 16 MOD 1 dives. Navy Experimental Diving Unit, Technical Report; NEDU TR 07-15, October 2007.
3) Clark, J. M. et al. Effects of prolonged oxygen exposure at 1.5, 2.0, or 2.5 ATA on pulmonary function in men (predictive studies V). Journal of applied physiology 86, 243-259 (1999)
4) Komarow H. D. et al. Impulse oscillometry in the evaluation of diseases of the airways in children. Ann Allergy Asthma Immunol. 2011 March; 106(3): 191-199
5) Demchenko, I. T., Zhilyaev, S. Y., Moskvin, A. N., Piantadosi, C. A. & Allen, B. W. Autonomic activation links CNS oxygen toxicity to acute cardiogenic pulmonary injury. Am J Physiol Lung Cell Mol Physiol 300, L102-111, doi: 10.1152/ajplung.00178.2010 (2011).
6) Lika Nesuashvili, S. H. H., Parmvir K. Bahia, and Thomas E. Taylor-Clark. Sensory Nerve Terminal Mitochondrial Dysfunction Activates Airway Sensory Nerves via Transient Receptor Potential (TRP) Channels. Molecular Pharmacology 83, 1007-1019 (2013).
7) Mark A. Birrell, P., Sara J. Bonvini, BSc, Eric Dubuis, PhD, Sarah A. Maher, PhD, Michael A. Wortley, PhD, Megan S. Grace, PhD, Kristof Raemdonck, PhD, John J. Adcock, PhD, and Maria G. Belvisi, PhD*. Tiotropium modulates transient receptor potential V1 (TRPV1) in airway sensory nerves: A beneficial off-target effect? The Journal of Allergy and Clinical Immunology 133, 679-687 (2014).
8) Clark, J. M. & Lambertsen, C. J. Rate of development of pulmonary O2 toxicity in man during 02 breathing at 2.0 Ata. J Appl Physiol 30, 739-752 (1971).
9) P. W. Holmes, C. E. B., R. J. Pierce. Chronic persistent cough: use of ipratropium bromide in undiagnosed cases following upper respiratory tract infection. Respiratory Medicine 86, 425-429 (1992).
10) Lambertsen, J. M. C. a. C. J. Rate of Development of Pulmonary O2 Toxicity in Man during 02 breathing at 2.0 Ata. Journal of Applied Physiology 30, 739-752 (1971).
11) Wellek, S. & Blettner, M. On the proper use of the crossover design in clinical trials: part 18 of a series on evaluation of scientific publications. Deutsches Arzteblatt international 109, 276-281, doi:10.3238/arztebl.2012.0276 (2012).
12) Brown, B. W., Jr. The crossover experiment for clinical trials. Biometrics 36, 69-79 (1980).
13) Birring, S. S. et al. Development of a symptom specific health status measure for patients with chronic cough: Leicester Cough Questionnaire (LCQ). Thorax 58, 339-343 (2003).
14) Stefan Wellek, a. M. B. On the Proper Use of the Crossover Design in Clinical Trials. Deutsches Arzteblatt 109, 276-281 (2012).

What is claimed is:

1) A method for preventing pulmonary oxygen toxicity due to oxygen exposures, comprises administering to a subject a prophylactically effective amount of an anticholinergic or pharmaceutically acceptable salts thereof, optionally together with a pharmaceutically acceptable excipient prior to an oxygen exposure.

2) The method according to claim 1, wherein the anticholinergic or pharmaceutically acceptable salts thereof is selected from a group consisting of tiotropium salts, oxitropium salts, flutropium salts, ipratropium salts, glycopyrronium salts and trospium salts.

3) The method according to claim 2, wherein said anticholinergic or pharmaceutically acceptable salts thereof is selected from a group consisting of tiotropium bromide, oxitropium bromide, glycopyrronium bromide and ipratropium bromide.

4) The method according to claim 1, wherein said anticholinergic or pharmaceutically acceptable salts thereof is administered in one or more dose prior to an oxygen exposure.

5) The method according to claim 4, wherein said anticholinergic or pharmaceutically acceptable salts thereof is administered within 24 hours prior to an oxygen exposure.

6) The method according to claim 1, wherein said anticholinergic or pharmaceutically acceptable salts thereof is administered via oral inhalation.

7) The method according to claim 1, wherein said anticholinergic or pharmaceutically acceptable salts thereof is administered prior to each of a plural of oxygen exposures.

8) The method according to claim 1, wherein said anticholinergic or pharmaceutically acceptable salts thereof is administered to prevent tracheobronchitis due to PO2T.

9) The method according to claim 1, wherein said anticholinergic or pharmaceutically acceptable salts is administered to prevent decrements in pulmonary function due to PO2T.

10) The method according to claim 1, wherein said pulmonary toxicity is caused by hyperbaric oxygen exposure.
